(19) United States
(12) Patent Application Publication
(10) Pub. No.: US 2026/0077169 A1
(43) Pub. Date: Mar. 19, 2026

(54) ORAL DEVICE FOR ACTIVE COMPONENT DELIVERY

(71) Applicant: FSV Solutions LLC, Waltham, MA (US)

(72) Inventors: Gary Fleischman, Waltham, MA (US); Khashayar Vakili, Waltham, MA (US)

(21) Appl. No.: 19/312,608

(22) Filed: Aug. 28, 2025

Related U.S. Application Data
(60) Provisional application No. 63/688,968, filed on Aug. 30, 2024.

(51) Int. Cl. A61M 31/00

(52) U.S. Cl. CPC A61M 31/002; A61M 2202/0007; A61M 2202/06

(57) ABSTRACT

An oral device or mouthguard device containing a reservoir for holding and passively releasing an active component is provided. An oral device or mouthguard device having a body with an inner wall, an outer wall, a floor spanning the inner wall and outer wall. The inner wall, the outer wall, and the floor define a channel including an interior channel surface and an exterior channel surface. The interior channel surface shaped to receive a plurality of teeth. A reservoir integral with the inner wall is also included. The reservoir has a roof portion and a base portion defining an internal volume for an active component. The roof portion has an opening for receiving the active component and the base portion has one or more openings.

### CROSS-REFERENCE TO RELATED APPLICATIONS

This application claims the benefit of U.S. Provisional App. No. 63/688,968, filed on Aug. 30, 2024. The entire contents of which are hereby incorporated by reference.

### TECHNICAL FIELD

The disclosure is generally directed to oral devices or mouthguard devices to deliver an active component to the mouth or throat of a user.

### BACKGROUND OF THE DISCLOSURE

Ailments, such as sore throats, mouth sores, mouth ulcers, or post-surgical oropharyngeal pain can cause discomfort or pain, which can subsequently affect sleep. Moreover, the irritation caused by sore throats, for example, can increase snoring or coughing, further disrupting sleep patterns. Active components such as lozenges, gels, or sprays that incorporate numbing substances such as benzocaine or phenol are commercially available for addressing the pain or discomfort caused by sore throats and mouth ulcers. However, each of these options comes with its own set of concerns. For example, there is a potential risk of choking when using lozenges during sleep. This may necessitate uncomfortable adjustments to head positions in order to mitigate this risk. Furthermore, numbing agents fail to address commonly experienced throat dryness, often leading to episodes of coughing and frequent disturbances in sleep patterns. Similarly, gels or ointments that decrease the pain associated with mouth ailments wear off in a short period of time and are not as long lasting.

### SUMMARY

The purpose and advantages of the disclosed subject matter will be set forth and apparent from the description that follows, as well as will be learned by practice of the disclosed subject matter. Additional advantages of the disclosed subject matter will be realized and attained by the devices particularly pointed out in the written description and claims hereof, as well as from the appended drawings.

In various embodiments, the present disclosure is directed to oral devices or mouthguard devices containing a reservoir for holding and passively releasing an active component.

To achieve these and other advantages and in accordance with the purpose of the disclosed subject matter, as embodied and broadly described, in some embodiments the disclosed subject matter includes an oral device or mouthguard device comprising a body comprising an inner wall, an outer wall, a floor spanning the inner wall and outer wall. The inner wall, the outer wall, and the floor can define a channel. The channel can include an interior channel surface and an exterior channel surface. The interior channel surface can be shaped to receive a plurality of teeth. The oral device or mouthguard device can further comprise a reservoir disposed on the exterior channel surface. The reservoir can define an interior volume for an active component. The reservoir can include a porous semipermeable membrane and an opening. The reservoir can include a holding element (e.g., an element disposed to seal the opening when engaged or a means for providing mechanical pressure to the opposing sides of the opening together).

In some embodiments the disclosed subject matter includes an oral device or mouthguard device comprising a body comprising an inner wall, an outer wall, and a floor spanning the inner wall and outer wall. The inner wall, the outer wall, and the floor can define a channel. The channel can include an interior channel surface and an exterior channel surface. The interior channel surface can be shaped to receive a plurality of teeth. The oral device or mouthguard device can further comprise a reservoir a reservoir integral with the inner wall. The reservoir can comprise a roof portion and a base portion defining an internal volume for an active component. The roof portion can comprise an opening for receiving the active component and the base portion comprising one or more openings.

In some embodiments, the active component further comprises a solid material. In some embodiments, the solid material further comprises a lozenge.

In some embodiments, the active component further comprises a gelatinous material. In some embodiments, the gelatinous material can further comprise an antiseptic gel.

In some embodiments, the active component further comprises a liquid material.

In some embodiments, the reservoir is a plurality of reservoirs

In some embodiments, the exterior channel surface defines a groove.

In some embodiments, the reservoir has a modular attachment to connect with the groove.

In some embodiments, the channel is shaped to receive the upper teeth of the wearer.

In some embodiments, the channel is shaped to receive the lower teeth of the wearer.

In some embodiments, the channel is heat moldable to the teeth of the wearer.

In some embodiments, the porous semipermeable membrane is formed of a material that allows fluid communication between the active component and salivary fluid.

In some embodiments, the porous semipermeable membrane comprises a mesh.

In some embodiments, the reservoir is formed of a semi-rigid material

In some embodiments, the channel is formed of a semi-rigid material or thermoplastic.

In some embodiments, the channel is shaped in a U-shaped arch.

In some embodiments, the reservoir extends from a portion of the inner wall.

In some embodiments, the reservoir is shaped and sized to cover at least a portion of the roof of the mouth of the wearer.

In some embodiments, the reservoir is shaped and sized to cover at least a portion of the area below the tongue of the wearer.

In some embodiments, the portion is a first portion, and the reservoir extends from the first portion of the inner wall to a second portion of the inner wall.

In some embodiments, the reservoir is disposed on a suspension mesh, the suspension mesh connected on the exterior channel surface from a first portion of the inner wall to a second portion of the inner wall.

In some embodiments, the holding element comprises a seal.

In some embodiments, the holding element comprises a means for providing mechanical pressure to the opposing sides of the opening together.

The disclosed subject matter also includes an oral device or mouthguard device comprising a body comprising an inner wall, an outer wall, a floor spanning the inner wall and outer wall. The inner wall, the outer wall, and the floor can define a channel. The channel can include an interior channel surface and an exterior channel surface. The interior channel surface can be shaped to receive a plurality of teeth. The oral device or mouthguard device can further comprise reservoir comprising a first suspension layer connected on the exterior channel surface from a first portion of the inner wall to a second portion of the inner wall. The first layer can define an interior volume for an active component. The first layer can be a porous semipermeable membrane and an opening.

In some embodiments, the reservoir is shaped and sized to cover at least a portion of the roof of the mouth of the wearer.

In some embodiments, the reservoir is shaped and sized to cover at least a portion of the area below the tongue of the wearer.

In some embodiments, the first layer defines an interior volume between a roof of the mouth and the first layer.

In some embodiments, the reservoir can further comprise a second layer. The second layer can connect on the exterior channel surface from the first portion of the inner wall to the second portion of the inner wall. The first layer and the second layer can define an opening for receiving an active component.

In some embodiments, the second layer is disposed to be about parallel to the first layer.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments. It is to be understood that in some instances various aspects of the described embodiments may be shown exaggerated or enlarged to facilitate an understanding of the described embodiments. In the drawing, like reference characters generally refer to like features, functionally, and/or structurally similar elements throughout the various drawings. The drawings are not necessarily to scale, emphasis instead being placed upon illustrating the principles of the teachings. The drawings are not intended to limit the scope of the present teachings in any way.

FIG. 1A illustrates a top view of an embodiment of a mouthguard, in accordance with embodiments of this disclosure.

FIG. 1B illustrates a perspective view of an embodiment of a mouthguard as shown in FIG. 1A, in accordance with embodiments of this disclosure.

FIG. 2A illustrates a side view of an embodiment of a reservoir, in accordance with embodiments of this disclosure.

FIG. 2B illustrates a partial cross-sectional view of an embodiment of a mouthguard, in accordance with embodiments of this disclosure.

FIG. 2C illustrates a side view of an embodiment of a reservoir, in accordance with embodiments of this disclosure.

FIG. 2D illustrates a partial cross-sectional view of an embodiment of a mouthguard, in accordance with embodiments of this disclosure.

FIG. 3 illustrates a bottom view of an embodiment of a mouthguard as shown in FIG. 1, in accordance with embodiments of this disclosure.

FIG. 4 illustrates a top view of an embodiment of a mouthguard, in accordance with embodiments of this disclosure.

FIG. 5 illustrates a top view of an embodiment of a mouthguard as shown in FIG. 4, in accordance with embodiments of this disclosure.

FIG. 6 illustrates a perspective view of an embodiment of a mouthguard as shown in FIG. 4, in accordance with embodiments of this disclosure.

FIG. 7A illustrates a top view of an embodiment of a mouthguard, in accordance with embodiments of this disclosure.

FIG. 7B illustrates a bottom view of a mouthguard as shown in FIG. 7A, in accordance with embodiments of this disclosure.

FIG. 7C illustrates a side view of a mouthguard as shown in FIG. 7A, in accordance with embodiments of this disclosure.

FIG. 7D illustrates a cross-sectional view of a mouthguard as shown in FIG. 7A, in accordance with embodiments of this disclosure.

FIG. 7E illustrates a side view of a mouthguard as shown in FIG. 7A, in accordance with embodiments of this disclosure.

FIGS. 8A and 8B illustrate perspective views of a mouthguard, in accordance with embodiments of this disclosure.

FIGS. 9A and 9B illustrate perspective views of various mouthguards, in accordance with embodiments of this disclosure.

FIGS. 10A and 10B illustrate perspective views of various mouthguards, in accordance with embodiments of this disclosure.

FIGS. 11A and 11B illustrate a perspective view and a side view, respectively, of a mouthguard positioned in a user's mouth, in accordance with embodiments of this disclosure.

### DETAILED DESCRIPTION

The various concepts introduced above and discussed in greater detail below may be implemented in a number of ways, as the described concepts are not limited to any particular manner of embodiment.

Reference will now be made in detail to the exemplary embodiments of the present disclosure, examples of which are illustrated in the accompanying drawings. Wherever possible, the same reference numbers will be used throughout the drawings to refer to the same or like parts.

References herein to positions of elements (e.g., “top”, “bottom”) are merely used to describe the orientation of various elements in the FIGURES. It should be noted that the orientation of various elements may differ according to other embodiments, and that such variations are intended to be encompassed by the present disclosure.

The term “about” means a range of values inclusive of the specified value that a person of ordinary skill in the art would reasonably consider to be comparable to the specified value. In some embodiments, “about” means within a standard deviation using measurements generally accepted by a person of ordinary skill in the art. In some embodiments, “about” means ranging up to ±10% of the specified value. In some embodiments, “about” means ranging up to ±5% of the specified value. In some embodiments, “about”means the specified value.

The present disclosure is directed to oral devices or mouthguard devices to deliver an active component to the mouth or throat (e.g., pharynx) of a user. The active components, can include, but are not limited to, analgesic agents for alleviating pain, anti-inflammatory agents for reducing swelling or irritation, and antimicrobial agents (e.g., antibiotics, antifungals, antivirals) for treating infections caused by bacteria, fungi, or viruses. The oral devices or mouthguard devices provided herein can be configured to administer one or more agents (e.g., therapeutic agents) to the oral cavity and/or the oropharyngeal region. In some implementations, the oral devices or mouthguard devices provided herein can be configured to deliver chemotherapeutic agents or other compounds for the treatment or management of cancers (e.g., oral and/or throat cancers) and/or precancerous lesions. In some embodiments, the oral devices or mouthguard devices provided herein can be configured to deliver agents for alleviating oral or esophageal pain or discomfort resulting from the treatment of cancer (e.g., chemotherapeutic side effects) In some implementations, the oral devices or mouthguard devices provided herein can be configured to treat various ailments, including but not limited to sore throats, mouth sores, mouth ulcers, or post-surgical oropharyngeal pain.

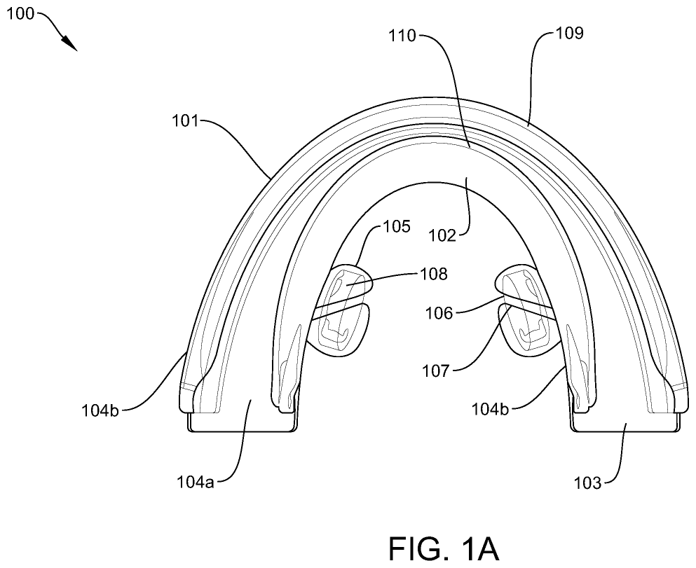

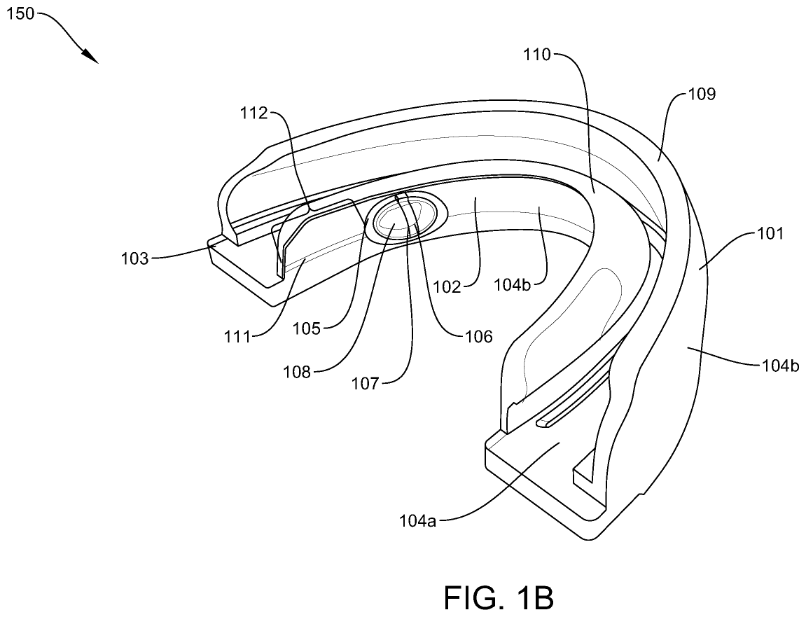

FIG. 1A is a diagram 100 illustrating a top view of a mouthguard according to an embodiment of the present disclosure. FIG. 1B is a diagram 150 illustrating a perspective view of a mouthguard according to an embodiment of the present disclosure. Referring now to FIGS. 1A-1B, the mouthguard can include an outer wall 101, an inner wall 102, a floor 103, and a reservoir 105. In various embodiments, the outer wall 101 and inner wall 102 are oriented vertically or about vertically (e.g., about a 90 degree angle from the floor) and spaced from each other such that the floor 103, being oriented perpendicularly with respect to the walls, spans the distance between the outer wall 101 and the inner wall 102. In some embodiments, the outer wall 101 and inner wall 102 are parallel. In this way, the outer wall 101, inner wall 102, and floor 103 can define a channel with an interior channel surface 104a and an exterior channel surface 104b. The interior channel surface 104a can be shaped and sized to receive a plurality of teeth (e.g., teeth of a mouth).

In some embodiments, the interior channel surface 104a is shaped to receive at least a portion of the upper teeth of the wearer. In some embodiments, the interior channel surface 104a is shaped to receive at least a portion of the lower teeth of the wearer. In some embodiments, the interior channel surface 104a is generally shaped and sized to receive the teeth of an adult. In some embodiments, the interior channel surface 104a is shaped and sized to receive the teeth of a child.

In some embodiments, the channel is generally a linear channel. In some embodiments, the channel is generally a U-shaped channel or U-shaped arch. By way of example, the channel can be curved to conform to the curve of the dental arch of the wearer (e.g., generic dental arch or custom-fitted to the dental arch of the specific wearer). In some embodiments, a custom-fitted channel can be formed using mold impression techniques. In some embodiments, the channel is heat moldable to the teeth of the wearer to create a custom fit. The channel can be formed of a semi-rigid material such as a thermoplastic.

In some embodiments, the reservoir 105 is integral with the channel. In some embodiments, the reservoir 105 is separate from the channel, for example, configured to be removably attachable to the channel.

Still referring to FIGS. 1A-1B, and in greater detail, the reservoir 105 can be disposed on the exterior channel surface 104b. The reservoir 105 can be sized and shaped to define an interior volume for holding an active component 108. The reservoir 105 can include a porous semipermeable membrane that allows fluid communication between the active component 108 and the salivary fluid of the wearer. In this way, the active component 108 can be passively released through pores of the semipermeable membrane of the reservoir 105. Salivary fluid can flow through the pores of the semipermeable membrane and into the reservoir 105, thereby allowing salivary fluid to mix with the active component 108. Subsequently, the mixture of salivary fluid and active component can exit the reservoir 105 through the pores of the semipermeable membrane. The flow of fluid into and out of the reservoir 105 is supported by the natural secretion of salivary fluid from the salivary glands of the wearer or user. The pores of the porous membrane can be shaped any of circular, rectangular, polygonal, or irregularly shaped. By way of example, the mixture of salivary fluid and active component can deliver the active component to the oral cavity and/or the oropharyngeal space (e.g., throat of the wearer), for instance as the wearer swallows or based on the orientation of the wearer's head. In another example, the mixture of salivary fluid and active component can be secreted out of the reservoir 105 such that it is secreted directly onto or proximally to a mouth ulcer. In another example, the mixture of salivary fluid and active component can bring the concentration of the active component to a therapeutic level and/or equilibrium in the wearer's mouth, such that it is applied to a portion of or all of the wearer's mouth.

The permeability of the porous semipermeable membrane of the reservoir 105 can be determined by the pore size, pore distribution, pore geometry, membrane thickness, material composition of the membrane, and/or surface chemistry of the membrane. The permeability of the porous semipermeable membrane of the reservoir 105 can determine the rate at which the active component 108 is released from the reservoir 105. In some embodiments, the porous semipermeable membrane has a degree of porosity sufficient to provide the desired permeability, and/or a pore size distribution within a range of about 0.01 mm to about 100 mm. In some embodiments, the average diameter of the pores of the membrane is between about 0.01 mm to about 100 mm. In some embodiments, the average diameter of the pores of the membrane is between about 0.01 mm to about 50 mm. In some embodiments, the average diameter of the pores of the membrane is between about 0.01 mm to about 30 mm.

In various embodiments, the reservoir 105 further includes an opening 106, and a holding element 107. In some embodiments, the holding element 107 is disposed to seal the opening 106 when engaged. An active component 108 can be inserted through the opening 106 of the reservoir. The holding element 107 can comprise a seal. The holding element 107 can subsequently be engaged to seal the opening 106 and the active component 108 is held within the reservoir 105. The active component 108 can be replaced by unsealing the holding element 107 and removing the loaded active component 108 and replacing the component with another active component. Alternatively, if the loaded active component 108 is depleted such that the reservoir 105 is empty, then the wearer can refill the reservoir 105 via the opening 106.

In some embodiments, the holding element 107 can include a means for providing mechanical pressure that pinches or compresses the opposing sides of the opening 106 together, thereby forming a seal through that mechanical pressure. The holding element 107 can be formed of a semi-rigid material such that the opening 106 can flex from a sealed to an open configuration during insertion of the active component 108. Once the active component 108 is inserted, the opening 106 can return to its sealed configuration, securely holding the active component 108 within the reservoir 105. In some embodiments, when the holding element 107 forms a seal through mechanical pressure, the sealed configuration does not include a physical seal to the opening 106. In some embodiments, when the holding element 107 forms a seal through mechanical pressure, the sealed configuration does include a physical seal to the opening 106.

In some embodiments, the active component 108 is a solid material, such as a lozenge or tablet (e.g., a dissolvable tablet). In some embodiments, the active component 108 is a gelatinous material, such as an antiseptic gel. In some embodiments, the viscosity of the gelatinous material is between about 100 cP and about 10,000 cP. In some embodiments, the active component 108 is a liquid material. In some embodiments, the viscosity of the liquid material is between about 1 cP and about 1,000 cP.

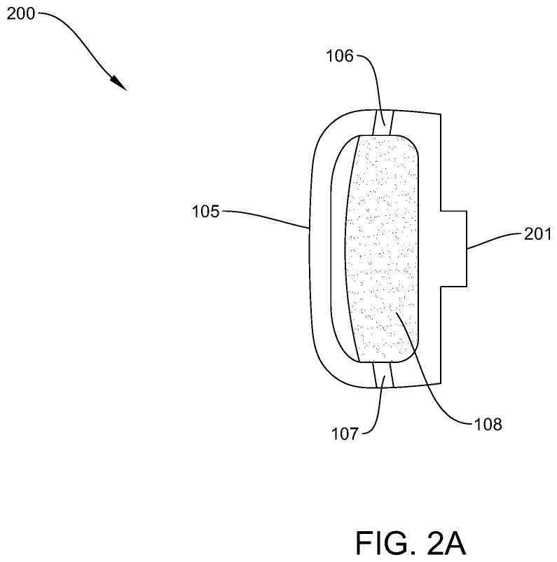

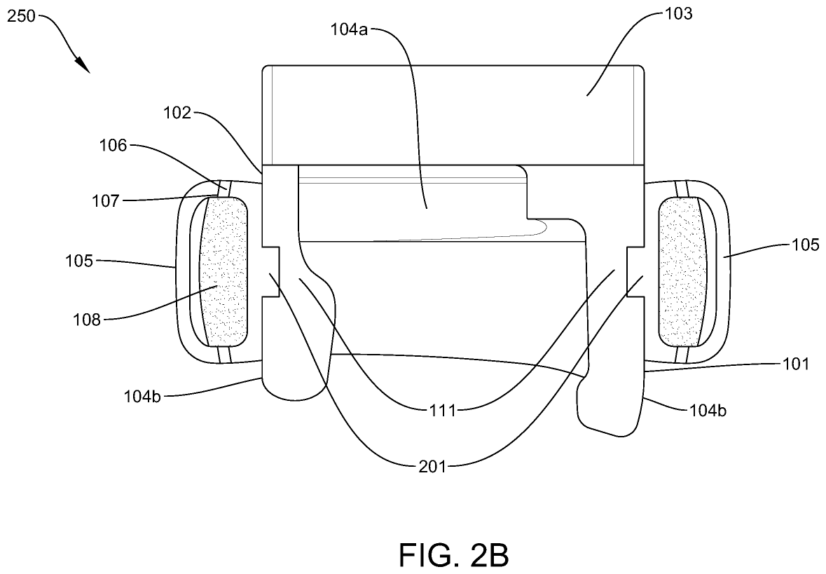

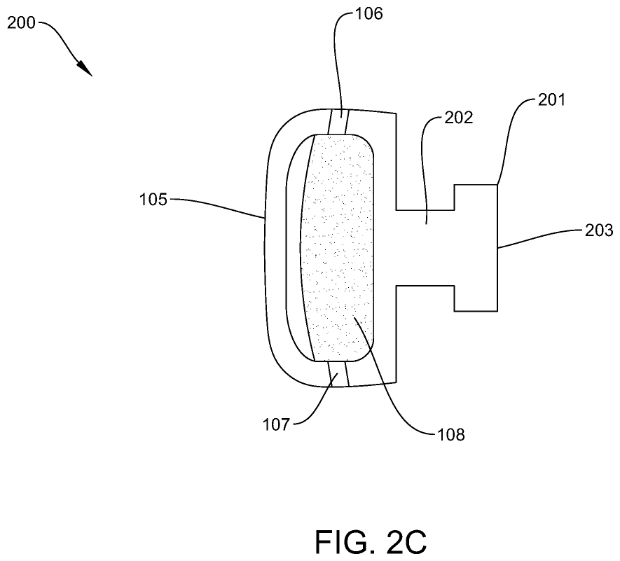

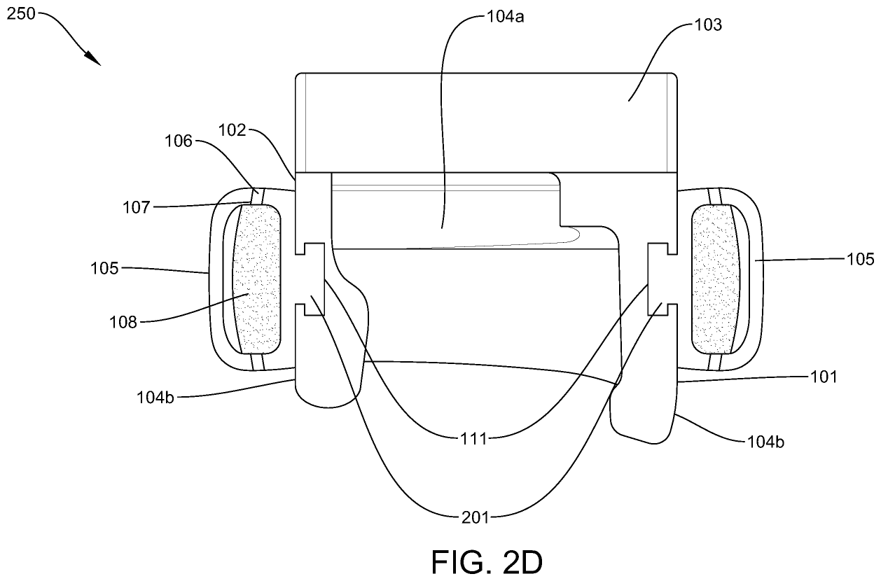

Still referring to FIGS. 1A-1B, with added reference to FIGS. 2A-2D, the reservoir 105 can further include a modular attachment 201 configured to connect with a groove 111 defined by the exterior channel surface 104b. The modular attachment 201 and the groove 111 can have corresponding shapes, such that the shape of the modular attachment 201 complements and aligns with the shape of the groove 111. In some embodiments, the modular attachment 201 is a flange extending from a surface of the reservoir 105. The flange can be shaped as a rectangular prism. The flange can be shaped as cylindrical prism. The friction between the interior surface of the groove 111 and the modular attachment 201 can provide a force that holds the reservoir 105 in place once it is positioned in the desired location along the groove 111, thereby preventing the reservoir 105 from sliding or detaching during use.

In some embodiments, the modular attachment 201 can be a flange comprising a first member 202 extending from a surface of the reservoir 105 and a second member 203 disposed at a free end of the first member 202 and substantially perpendicular to the first member 202 to form a T-shaped flange (illustrated in FIG. 2C). The second member 203 which forms a top portion of the T-shaped flange can engage with and fit into the groove 111 (illustrated in FIG. 2D). This arrangement can allow the modular attachment 201 to be securely mounted and positioned along the groove 111, thereby providing a stable and adjustable attachment.

In some embodiments, the groove 111 is a channel. In some embodiments, the groove 111 is a slot or a port. In some embodiments, the cross-sectional area of the groove 111 is substantially rectangular. In some embodiments, the cross-sectional area of the groove 111 is T-shaped.

In some embodiments, the reservoir 105 is permanently affixed to the exterior channel surface 104b. In some embodiments, the reservoir 105 extends from a first portion of the exterior channel surface 104b (e.g., the inner wall 101, the outer wall 102). In some embodiments, the reservoir 105 extends from a first portion of the inner wall 101 to a second portion of the inner wall 101. In some embodiments, the reservoir 105 can be sized and shaped to cover at least a portion of the roof of the mouth of the wearer. In some embodiments, the reservoir 105 can be sized and shaped to cover at least a portion of the area below the tongue of the wearer.

The modular attachment 201 and/or the groove 111 can include a locking feature designed to secure the modular attachment 201 in place once it is provided to a desired location along the groove 111. In some embodiments, the locking feature can secure the modular attachment 201 by sliding or placing the modular attachment 201 along the grove 111. By way of example, the modular attachment 201 can include one or more locking pins, tabs, or projections that engage with corresponding features in the groove 111 that, when engaged, prevent sliding or other movement of the modular attachment 201 when placed in the mouth, and when disengaged allow sliding or other movement of the modular attachment 201.

The groove 111 allows the wearer to slide, relocate, and/or remove the reservoir 105. The wearer can slide the reservoir 105 to a location along the groove 111 so that the release of the active component 108 is targeted to a specific or desired area of the mouth.

The length 112 of the groove 111 can vary. In some embodiments, the length 112 of the groove 111 spans the length of the wall (e.g., the outer wall 101, the inner wall 102) that defines the groove 111. In some embodiments, the length 112 of the groove 111 spans a portion of the length of the wall (e.g., the outer wall 101, the inner wall 102) that defines the groove 111.

One or more reservoirs can be disposed and distributed on the exterior channel surface. One or more reservoirs 105 can be disposed and distributed on the outer wall 101. In various embodiments, one or more reservoirs 105 can be disposed and distributed on the inner wall 102. One or more reservoirs 105 can be disposed and distributed on the floor 103. In various embodiments, one or more reservoirs 105 can be disposed and distributed on the inner wall 102 and the outer wall 101. It should be understood that various configurations are possible for positionally arranging one or more reservoirs 105. By way of example, FIG. 1A shows reservoirs 105 disposed and distributed on the inner wall 102. FIG. 2B illustrates a partial cross-sectional view of an embodiment of a mouthguard having reservoirs 105 disposed and distributed on the outer wall 101 and inner wall 102.

The reservoir 105 can be formed of a semi-rigid material. In some embodiments, the reservoir 105 can be the same material as the channel. In some embodiments, the reservoir 105 can be a different material from the channel. In some embodiments, the reservoir 105 is formed of a stretchable material.

In some embodiments, the reservoir 105 is disposed on a suspension mesh that is connected on the exterior channel surface 104b from a first portion of the inner wall 102 to a second portion of the inner wall 102.

Still referring to FIGS. 1A-1B, the top edge 109 of the outer wall 101 and the top edge 110 of the inner wall 102 can include a rounded edge. The rounded edge of the walls can help prevent irritation and discomfort against the gums compared to sharp edges which can dig into the soft tissue or gums of the mouth and cause discomfort or injury to the wearer. The rounded edges can also contribute to a better fit for the wearer.

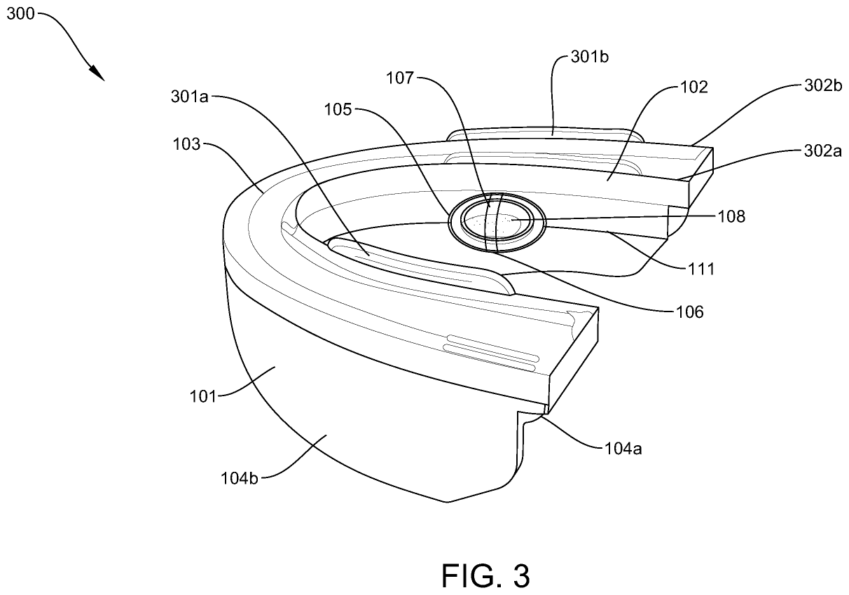

FIG. 3 is a diagram 300 illustrating a bottom view of a mouthguard as shown in FIG. 1 according to embodiments of the present disclosure. In some embodiments, the bottom of the mouthguard can further include one or more bumpers 301a,b. The bumper 301a,b can be a protruding edge that extends perpendicularly or about perpendicularly from the floor 103. In some embodiments, the bumper 301a,b can extend perpendicularly or about perpendicularly from the floor 103 along an edge 302a of the floor 103 such that the bumper 301a,b is parallel to the inner wall 102 (as illustrated in FIG. 3, bumper 301a). In some embodiments, the bumper 301a,b can extend perpendicularly or about perpendicularly from the floor 103 along an edge 302b of the floor 103 such that the bumper 301a,b is parallel to or about parallel the outer wall 101 (as illustrated in FIG. 3, bumper 301b). The bumper 301a,b can serve several purposes including: 1) provide additional cushioning and protection for the teeth, gums, and/or jaw of the wearer, 2) maintain the jaw in a stable position, 3) maintain the mouthguard in place and prevent it from shifting or dislodging.

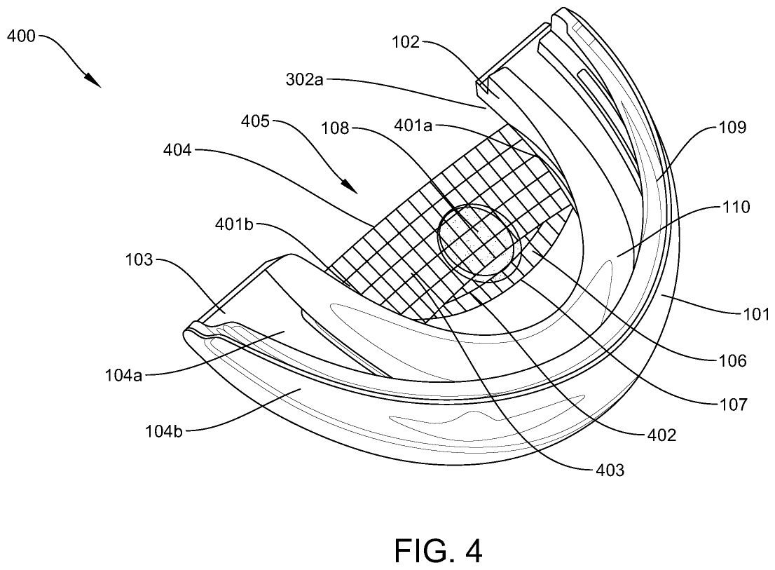

FIG. 4 is a diagram 400 of a perspective view of a mouthguard including an outer wall 101, an inner wall 102, a floor 103, a reservoir 405 including a first layer 402 and a second layer 403, according to embodiments of the present disclosure. In some embodiments, the first layer 402 and second layer 403 are formed of a semi-rigid material. In some embodiments, the first layer 402 and second layer 403 are formed of a material that is flexible and/or stretchable. In some embodiments, the reservoir 405 is integral with the channel (formed by the outer wall 101, inner wall 102, and floor 103). In some embodiments, the reservoir 405 is separate from the channel, for example, configured to be removably attachable to the channel. The outer wall 101 and inner wall 102 are oriented vertically and spaced from each other such that the floor 103, being oriented perpendicularly or about perpendicularly (e.g., at about a 90-degree angle) with respect to the walls, spans the distance between the outer wall 101 and the inner wall 102. In this way, the outer wall 101, inner wall 102, and floor 103 can define a channel with an interior channel surface 104a and an exterior channel surface 104b. The interior channel surface 104a can be shaped and sized to receive a plurality of teeth.

Still referring to FIG. 4, the first layer 402 can extend from a portion 401a, b of the inner wall 102 on the exterior channel surface 104b. The first layer 402 can extend from a first portion 401a of the inner wall 102 to a second portion 402b of the inner wall 102. The first layer 402 can define an interior volume (e.g., between itself and a surface of the mouth such as the roof of the mouth) which can hold an active component 108. The active component 108 can be held in place by the tensioning in the first layer 402. In some embodiments, a reservoir 105 containing an active component 108 can be inserted and held between the first layer 402 and the wearer's mouth. The reservoir 105 holding the active component 108 can be held in place by the tensioning in the first layer 402. A wearer can insert and position the mouthguard in the mouth. In following, the wearer can stretch the first layer 402 to insert either a reservoir 105 holding an active component 108 or the active component 108 alone.

In some embodiments, the first layer 402 is shaped and sized to cover at least a portion of the roof of the mouth of the wearer. For example, when the mouthguard is inserted into the mouth and the channel receives the upper teeth of the wearer, the first layer 402 is disposed between the roof of the mouth and tongue of the wearer. In some embodiments, the first layer 402 is shaped and sized to cover at least a portion of the area below the tongue of the wearer. For example, when the mouthguard is inserted into the mouth and the channel receives the lower teeth of the wearer, the first layer 402 is disposed below the tongue of the wearer.

In some embodiments, a second layer 403 can extend from the first portion 401a of the inner wall 102 to the second portion 402b of the inner wall 102 defining an interior volume between the first layer 402 and the second layer 403 for holding an active component 108. A pair of coinciding edges 404 of the layers 402, 403 can be coupled, creating a closed boundary of the interior volume. The edges of the layers opposing the closed boundary define an opening 106, which allows for insertion of an active component 108. In some embodiments, the first layer 402 and the second layer 403 extend from a first portion 402a of the inner wall 102.

The first portion 401a and the second portion 402b can be both the same vertical distance or about the same vertical distance from the edge 302a of the floor 103 such that they are aligned horizontally at the same level or about the same level. In some embodiments, the first portion 401a and second portion 401b are located proximal to the edge 302a of the floor 103. In some embodiments, the first portion 401a and second portion 401b are located distally from the edge 302a of the floor 103. In some embodiments, the first portion 401a and second portion 401b are located along the edge 302a of the floor 103.

In some embodiments, the first layer 402 and the second layer 403 are shaped and sized to cover at least a portion of the roof of the mouth of the wearer. For example, when the mouthguard is inserted into the mouth and the channel receives the upper teeth of the wearer, the first layer 402 and the second layer 403 are disposed between the roof of the mouth and tongue of the wearer. In some embodiments, the first layer 402 and the second layer 403 are shaped and sized to cover at least a portion of the area below the tongue of the wearer. For example, when the mouthguard is inserted into the mouth and the channel receives the lower teeth of the wearer, the first layer 402 and the second layer 403 are disposed below the tongue of the wearer.

The first layer 402 and second layer 403 can each include a porous semipermeable membrane that allows fluid communication between the active component 108 and the salivary fluid of the wearer. In this way, the active component 108 can be passively released through the pores of the semipermeable membrane of the layer. Salivary fluid can flow through the pores of the semipermeable membrane and through the pores of the layers 402,403, thereby allowing salivary fluid to mix with the active component 108. Subsequently, the mixture of salivary fluid and active component can exit the layers 402,403 through the pores of the semipermeable membrane. The flow of fluid into and out of the layers 402,403 of the reservoir can be supported by the natural secretion of salivary fluid from the salivary glands of the wearer. In some embodiments, each of the layers 402,403 includes a porous semipermeable membrane having multiple pores extending therethrough, the membrane having a degree of porosity sufficient to provide the desired permeability, and/or a pore size distribution within a range of about 0.01 mm to about 100 mm. In some embodiments, the average diameter of the pores of the membrane is between about 0.01 mm to about 100 mm. In some embodiments, the average diameter of the pores of the membrane is between about 0.01 mm to about 50 mm. In some embodiments, the average diameter of the pores of the membrane is between about 0.01 mm to about 30 mm. In some embodiments, the pores are arranged in a grid-like pattern, such as a rectangular array of pores spaced apart along orthogonal axis (e.g., as shown in FIGS. 4-6) The pores of the porous membrane can be different shapes including circular, rectangular, polygonal, or irregularly shape. In some embodiments, the porous semipermeable membrane includes a mesh. In some embodiments, each of the layers 402, 403 includes a mesh having a plurality of discrete openings defined by intersecting elements (e.g., strands, filaments, or etched ribs) arranged in a pattern, such as a grid or a lattice.

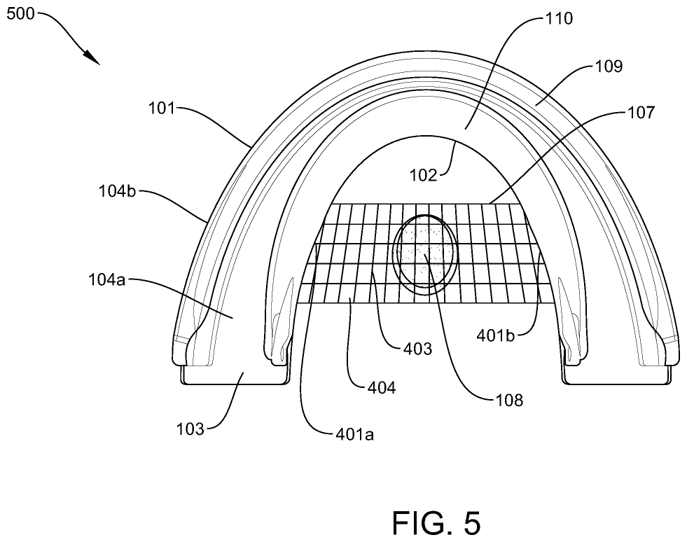

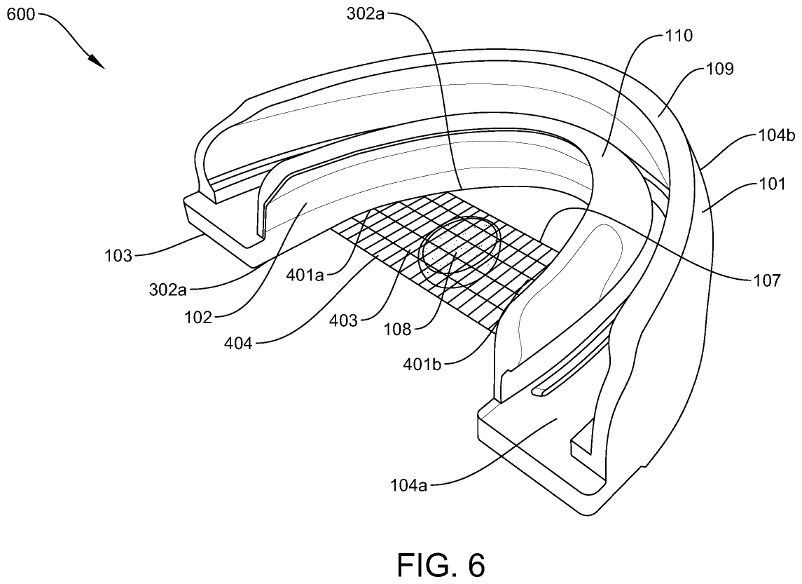

FIGS. 5-6 illustrate a top view (diagram 500) and perspective view (diagram 600), respectively, of an embodiment of a mouthguard, as shown in FIG. 4, in accordance with embodiments of this disclosure. These diagrams illustrate the mouthguard with the holding element 107 engaged according to embodiments of the present disclosure. The holding element 107 may involve a method of pinching or compressing the opposing sides of the opening 106 together, thereby creating a seal through mechanical pressure.

In various embodiments, a holding element 107 is disposed to seal the opening 106 when engaged. An active component 108 can be inserted through the opening 106. The holding element 107 can subsequently be engaged to seal the opening 106 and the active component 108 is held between the layers 402,403 of the reservoir. The active component 108 can be replaced by unsealing the holding element 107 and removing the loaded active component 108 and replacing the component with another active component. Alternatively, if the loaded active component 108 is depleted, then the wearer can refill the interior volume between the layers 402,403 via the opening 106. In some embodiments, the wearer can load the active component 108 while wearing the mouthguard. In some embodiments, the wearer can load the active component 108 before inserting and positioning the mouthguard within the mouth. The active component 108 can be held in place between the layers 402,403 through the tensioning of the layers, as well as by the friction between the active component 108 and the surfaces of the layers 402,403.

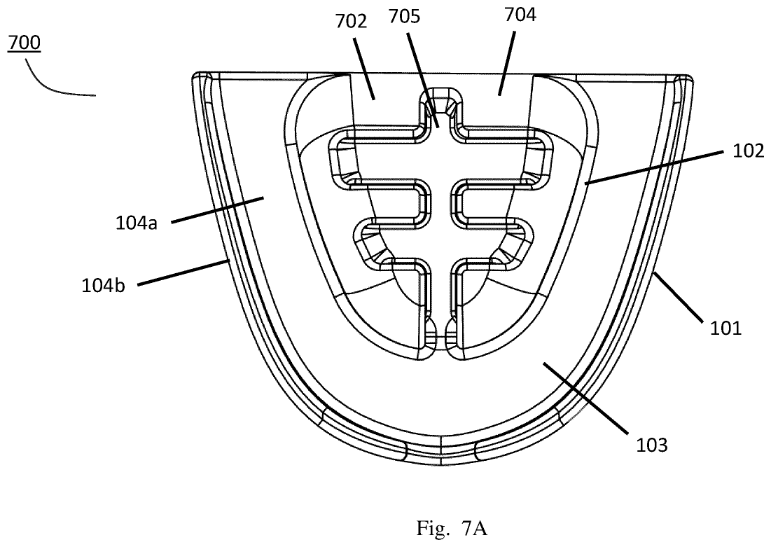

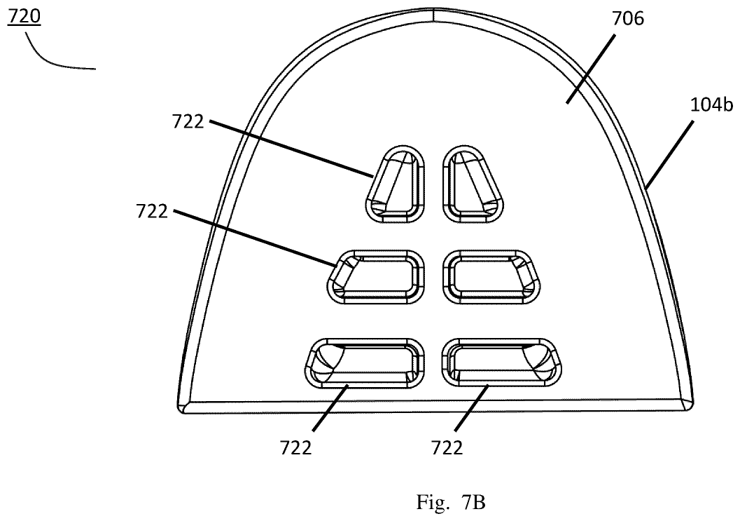

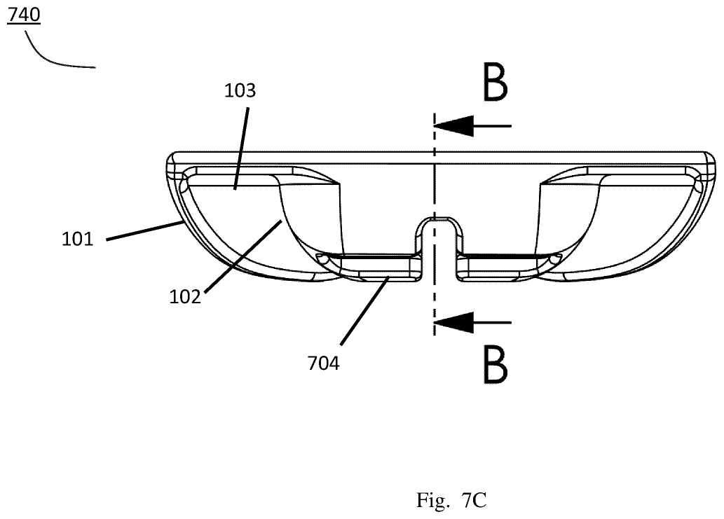

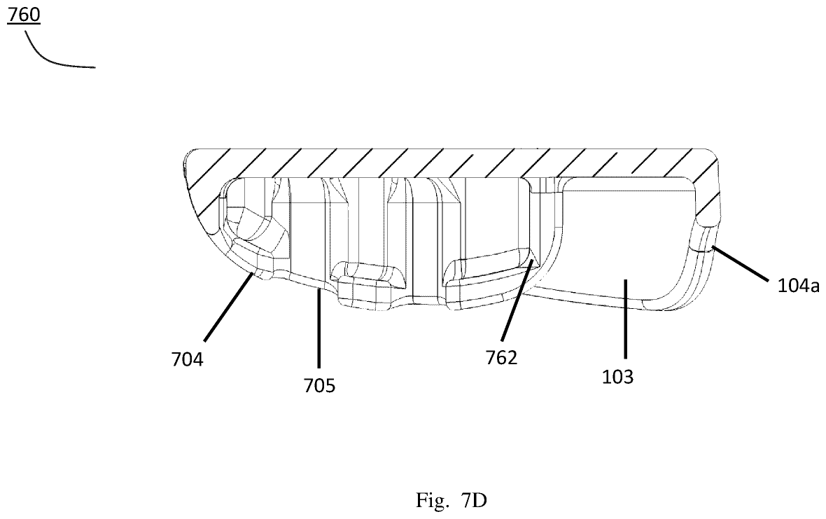

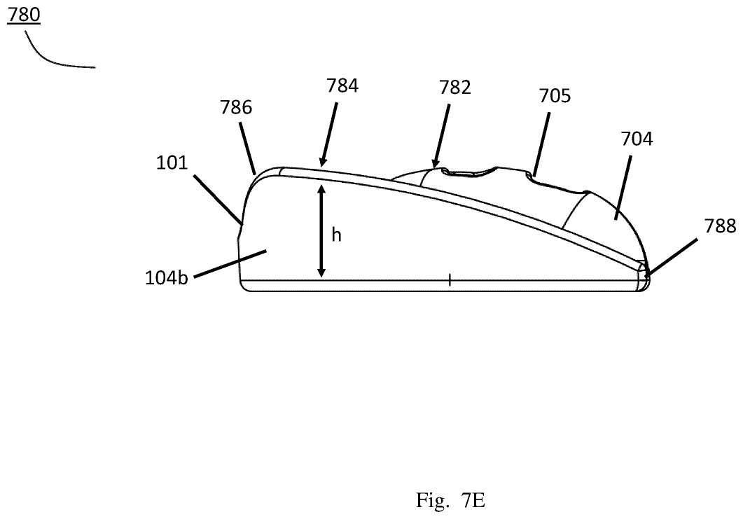

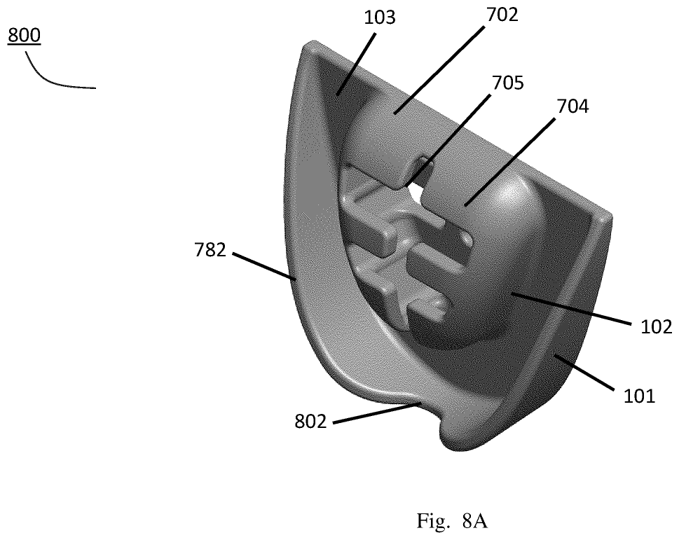

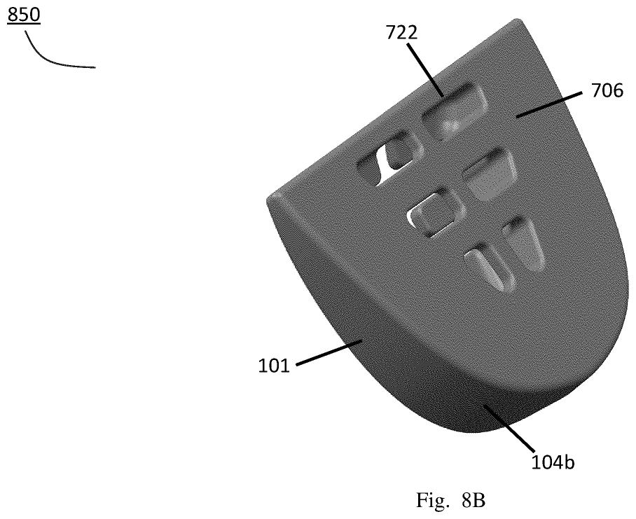

FIGS. 7A and 7B illustrate a top view (diagram 700) and a bottom view (diagram 720), respectively, of an embodiment of a mouthguard including an outer wall 101, an inner wall 102, a floor 103, and a reservoir 705 according to embodiments of the present disclosure. FIG. 7C illustrates a side view (diagram 740) of an embodiment of a mouthguard, as shown in FIGS. 7A and 7B, in accordance with embodiments of this disclosure. FIG. 7D illustrates a cross-sectional view (diagram 760) of the mouthguard taken through the annotated region B in FIG. 7C. FIG. 7E illustrates an orthogonal side view (diagram 780) of a mouthguard, as shown in FIGS. 7A and 7B, in accordance with embodiments of this disclosure. FIGS. 8A and 8B illustrate perspective views (diagram 800, 850) of an embodiment of a mouthguard, as shown in FIGS. 7A and 7B. As shown in FIGS. 7A-8B , the outer wall 101, inner wall 102, and floor 103 can be configured to define a channel with an interior channel surface 104a and an exterior channel surface 104b. The interior channel surface 104a can be shaped and sized to receive a plurality of teeth. The reservoir 702 can be integral with the inner wall 102 and can be shaped and sized to receive the active component 108. In some embodiments, the outer wall 101 and inner wall 102 are disposed in parallel with respect to one another and perpendicular to the floor 103. In some embodiments, the outer wall 101 and inner wall 102 are not disposed in parallel with respect to one another. For example, the inner wall 102 can be oriented perpendicular to the floor 103 and the outer wall 101 can be disposed at an angle (e.g., inclined inwardly towards the floor 103).

The mouthguard (e.g., including the channel and reservoir 702) can be formed as a solid body, such as a molded or 3D-printed component. The body of the mouthguard can be formed of a biocompatible, flexible material such as silicone, thermoplastic elastomer (TPE), or like material. In some embodiments, the mouthguard is formed of a transparent (e.g., optically clear) material. In this way, a wearer can visually inspect the placement of the active component 108 within the mouthguard to ensure proper positioning prior to or during use. In some embodiments, the exterior surface of the mouthguard includes a polished finish, providing a smooth surface, thereby reducing friction against oral tissues and minimizing buildup of debris (e.g., food, bacteria).

The reservoir 702 can include an access region through which an active component 108 can be inserted into the internal holding region of the reservoir, and a release region configured to allow for the release or diffusion of the active component (e.g., active agent) into the oral cavity and/or oropharyngeal space. The geometry and location of the access region and release region can be adapted based on the form of the active component (e.g., solid, gel, or liquid) or based on the ailment to be treated. In some embodiments, the access region includes a stretchable or deformable opening to facilitate insertion or removal of the active component, while the release region includes one or more regions to allow interaction with salivary or delivery of the component's contents.

The reservoir 702 can include a roof portion 704 and base portion 706 that defined opposing surfaces of the reservoir and together enclose an interior volume or holding region configured to receive and retain the active component 108. The access region can be formed in the roof portion 706, while the release region can be defined in the base portion 706. The reservoir 702 can be bounded on a first (top) side by the base portion 706, on a second (bottom) side by the roof portion 704, and laterally by the inner wall 102. In some embodiments, the base portion 706 and roof portion 704 are integrally formed with the inner wall 102 such that each portion is contiguous with the channel (defined by the outer wall 101, inner wall 102, and floor 103). The reservoir 702 can be structurally continuous with the inner wall 102 around at least a portion of the wall's perimeter. The roof portion 704 can have a lower peak height compared to the uppermost edge of the outer wall 101.

The reservoir 702 can include a porous semipermeable membrane that allows fluid communication between the active component 108 disposed therein and the salivary fluid of the wearer. In this way, the active component 108 can be passively released through pores of the semipermeable membrane of the reservoir 702. In some embodiments, the porous semipermeable membrane has a degree of porosity sufficient to provide the desired permeability, and/or a pore size distribution within a range of about 0.01 mm to about 100 mm. In some embodiments, the average diameter of the pores of the membrane is between about 0.01 mm to about 100 mm. In some embodiments, the average diameter of the pores of the membrane is between about 0.01 mm to about 50 mm. In some embodiments, the average diameter of the pores of the membrane is between about 0.01 mm to about 30 mm.

The base portion 706 can include one or more openings 722 form through its surface. In some embodiments, the one or more openings 722 are distributed in a grid-like pattern. The one or more openings 722 can vary in shape and size; for example, the one or more openings 722 can be generally elongated or polygonal, with rounded or chamfered edges. In some embodiments, the one or more openings 722 are aligned in rows and arranged in a pattern that is symmetric across the sagittal plane of the mouthguard (a plane bisecting the mouthguard into symmetric halves). Each opening 722 can define a passage through which salivary fluid can flow, allowing for bidirectional fluid exchange between the reservoir and the oral cavity and/or oropharyngeal space. The size and spacing of the one or more openings 722 can be selected to balance retention of the active component 108 while permitting diffusion of dissolved agents.

The roof portion 704 can include an opening 705 through which the active component 108 can be inserted into the cavity. The opening 705 of the roof portion 704 can be configured to receive an active component 108 that is solid or gel-based. In some embodiments, the opening 705 is a circular or elliptical aperture. In some embodiments, the opening 705 is branched or slotted in shape (e.g., a cross-slit, star-shape, or multi-flap configuration). In some aspects, the shape of the opening 705 can facilitate guided insertion of the active component 108 and passive retention, while allowing the surrounding material to flex during placement or removal of the active component. The opening 705 can temporarily stretch or deform to accommodate insertion or removal of the active component 108 and return to its original shape. In some embodiments, the opening 705 includes sidewalls having a draft angle 762 (e.g., as shown in FIG. 7D). The draft angle 762 can taper the interior walls of the opening 705, resulting in interior walls that widen or narrow along the depth of the opening. In some embodiments, the edges of the opening 705 are rounded or beveled.

In some embodiments, the roof portion 704 and the base portion 706 differ in their geometry to complement their respective anatomical placement within the mouth of the wearer. For example, the roof portion 704 can be configured to resemble the curvature of the roof of the mouth. The roof portion 704 can have a curved profile 782 that follows a convex contour, extending upwardly from the floor 103 in a dome shape or arched shape. This curvature can allow the roof portion 704 to conform to the palatal vault of the wearer's mouth. The base portion 706 can be disposed along the bottom surface of the mouthguard (e.g., on the underside of the floor 103), positioned to be directly adjacent to the tongue of the wearer when the mouthguard is worn. In some embodiments, the base portion 706 is planar. In some embodiments, the base portion 706 is concave (e.g., curved inward toward the internal cavity of the mouthguard) or recessed relative to the surrounding floor 103. In some aspects, this can preserve tongue space and minimize interference with speech.

As shown in FIG. 7E, the outer wall 101 can extend upwardly from the floor 103 and include a contoured profile 784 (e.g., tapered profile, undulating profile). The outer wall 101 can have a non-uniform height profile. For example, the height (annotated “h” in FIG. 7E) of the outer wall 101 can be greatest at or near a sagittal plane of the mouthguard, corresponding to the anterior central region 786 of the dental arch, and gradually decrease toward the rear region 788, where it terminates at a lower height. In some embodiments, the outer wall 101 includes a localized dip 802 (depression in height) at or near the sagittal plane of the mouthguard (e.g., as shown in FIG. 8A). The localized dip 802 (e.g., notch) can be disposed between portions of the outer wall 101 (along the contoured profile 784) having a greater height. In some aspects, such a localized dip can provide clearance for the labial frenulum or upper lip tissue.

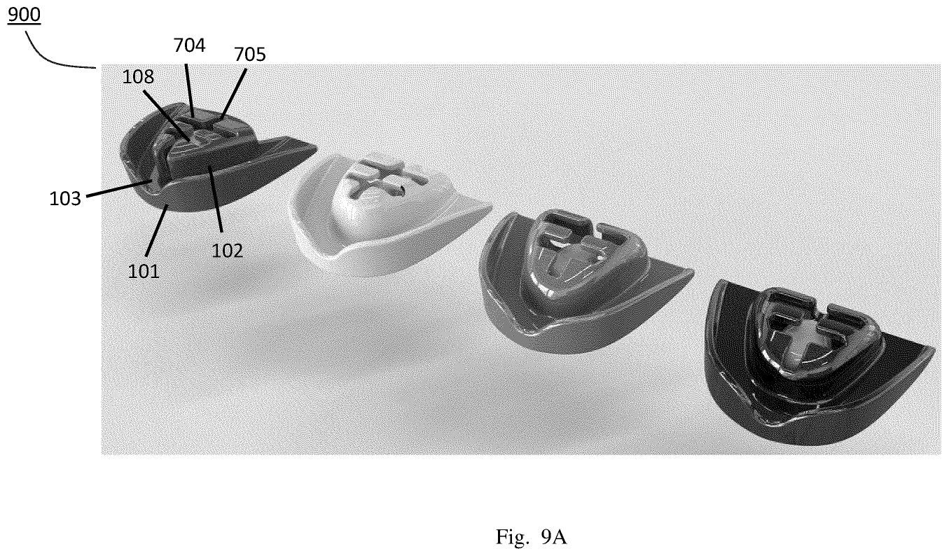

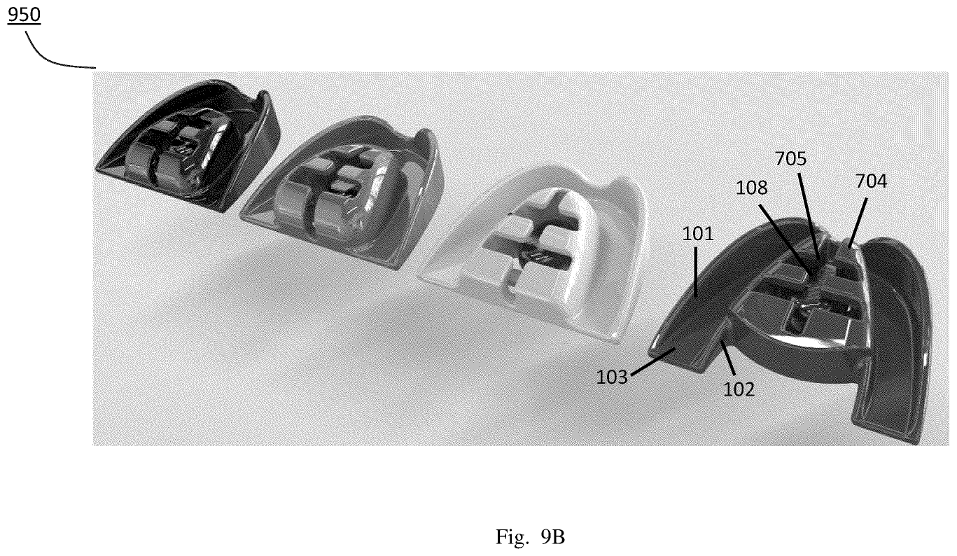

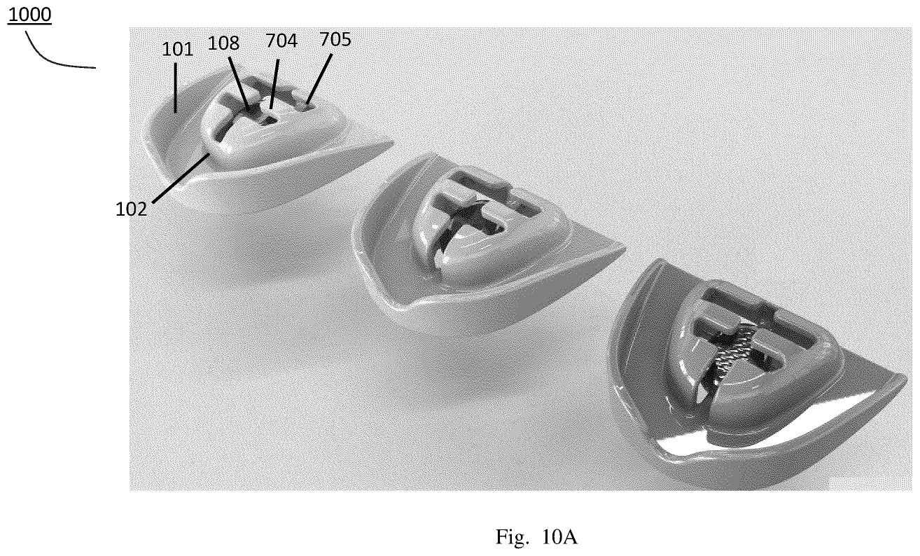

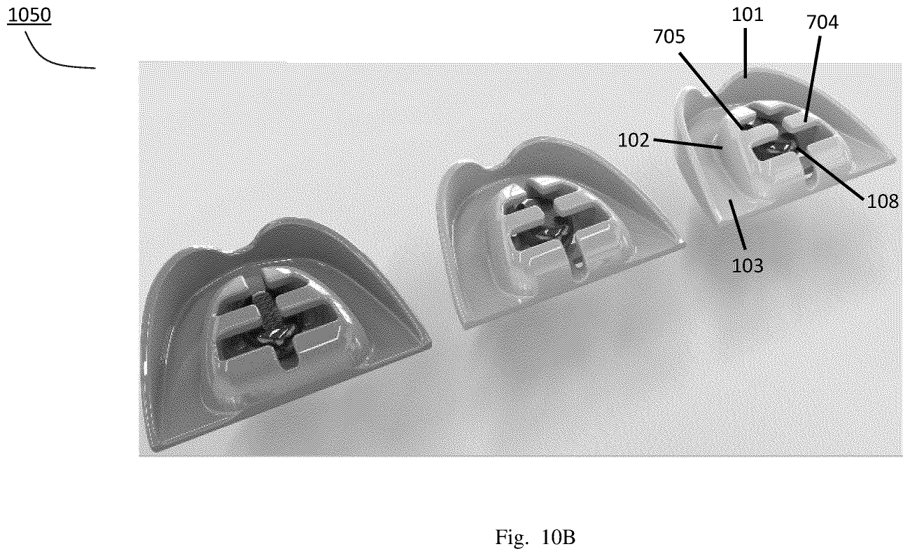

FIGS. 9A-10B illustrate perspective views (diagrams 900, 950, 1000, 1050) of various embodiments of a mouthguard, according to embodiments of the present disclosure. As shown in FIGS. 9A-10B , the shape and size of the mouthguard (e.g., geometry of the roof portion 704, base portion 706) can be optimized for different anatomical and functional contexts. These structural variations can optimize different aspects of device performance, including: fit and comfort when worn; ease of deployment and replacement of the active component; salivary access to the interior of the pocket; and manufacturability of the mouthguard, such as moldability and material flow.

For example, a reservoir 702 formed defined between the base portion 706 and roof portion 704 can vary in depth, width, or elevation relative to the floor 103. The roof portion 704 can differ in contour to match the user's palatal anatomy. An opening 705 of the roof portion 704 can be circular, elliptical, slotted, or branched, enabling insertion of solid active components or injection of gel or fluid based active components. The circular opening can provide a uniform or almost uniform aperture that facilitates easy insertion of tablet-form or lozenge-type active components while maintaining structural integrity of the roof portion 704. An elliptical opening can offer directional insertion guidance, with the longer axis oriented to accommodate elongated solid components or to provide a larger cross-sectional area for viscous gel insertion. Slotted openings can include linear cuts or cross-shaped incisions that provide retention of the active component. Branched openings, such as multi-flap configurations, can provide multiple insertion pathways and enhanced sealing characteristics, where each branch can flex independently to accommodate different component geometries while maintaining a secure closure around the inserted active component. The opening 705 can be sized to create a slight interference fit with the active component, ensuring retention within the interior volume while allowing for controlled release through the opening 705. In some embodiments, the edges of the opening 705 include rounded or chamfered surfaces to prevent tearing of the material during repeated insertion and removal cycles.

When the mouthguard is positioned in the user's mouth, the opening 705 may be disposed against the roof of the mouth, allowing the active component to be dispensed through the opening 722 into the oral cavity. The opening 705 may include one or more cavities that are sized such that the active component does not fit through those cavities in its initial form. In some embodiments, the one or more cavities may be configured to allow portions of the active component to pass through once the component has dissolved, degraded, or otherwise reduced to a safe size that does not present a choking hazard to the wearer. This configuration may provide a safety mechanism that prevents large pieces of the active component from being released into the mouth while allowing therapeutic agents to be delivered as the component breaks down to smaller, safer dimensions.

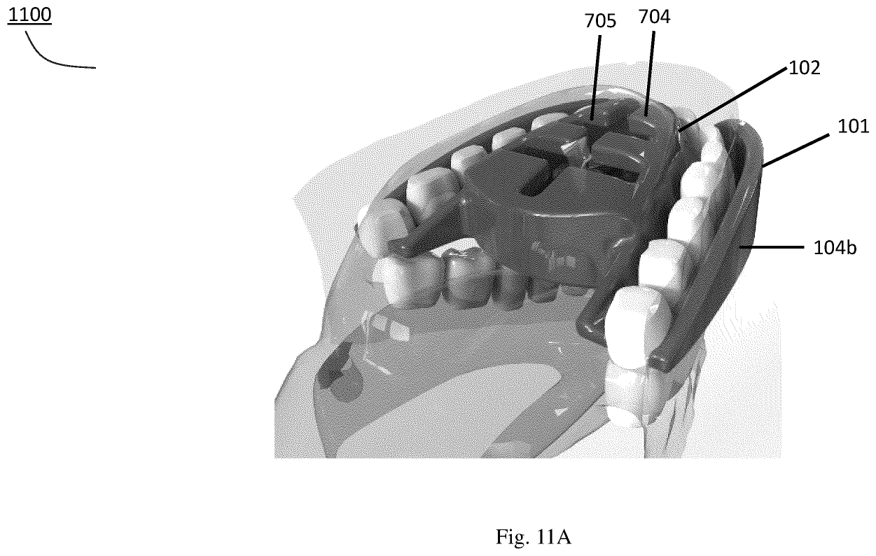

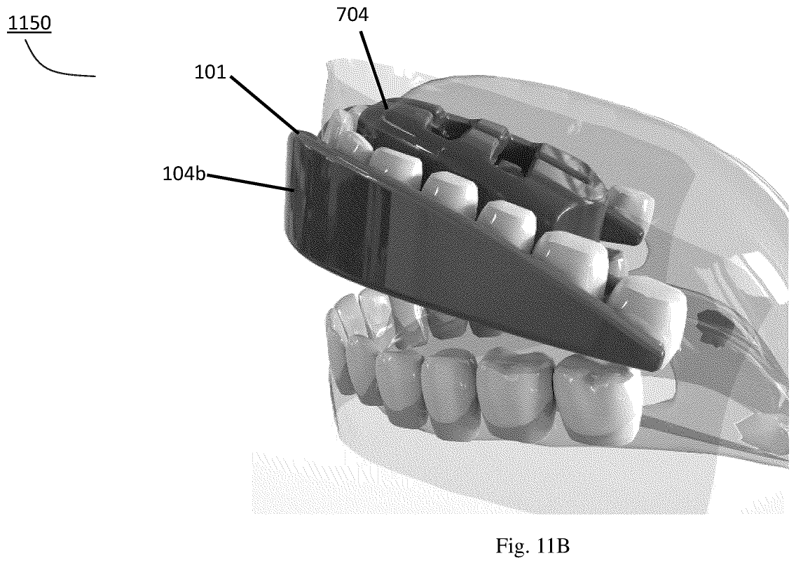

FIGS. 11A and 11B illustrate a rear perspective view (diagram 1100) and a side view (diagram 1150), respectively, of an embodiment of a mouthguard retained along the upper teeth, with the exterior channel 104b and outer wall 101 extending along the labial and buccal surfaces. The mouthguard can be retained in the mouth such that it minimizes interference with speech or natural movement of the tongue. The mouthguard can be passively retained by conformal engagement with the dental arch and can optionally include features to enhance frictional or suction-based retention (e.g., along the palatal and labial surfaces).

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. While some examples presented herein involve specific combinations of functions or structural elements, it should be understood that those functions and elements may be combined in other ways according to the present disclosure to accomplish the same or different objectives. Elements, components, and features discussed in connection with one embodiment are not intended to be excluded from other embodiments. Additionally, elements, components, and features described herein may be further divided into additional components or joined together to form fewer components for performing the same or similar functions. The terminology used herein was chosen to best explain the principles of the embodiments, the practical application or technical improvement over technologies found in the marketplace, or to enable others of ordinary skill in the art to understand the embodiments disclosed herein.

What is claimed:

1. A mouthguard comprising:
a body comprising:
an inner wall,
an outer wall,
a floor spanning the inner wall and outer wall, wherein the inner wall, the outer wall, and the floor define a channel, the channel including an interior channel surface and an exterior channel surface,
the interior channel surface shaped to receive a plurality of teeth; and
a reservoir disposed on the exterior channel surface,
the reservoir defining an interior volume for an active component;
the reservoir including a porous semipermeable membrane and an opening, wherein the reservoir includes a holding element.

2. The mouthguard of claim 1, wherein the active component further comprises a solid material, a gelatinous material, or a liquid material.

3. The mouthguard of claim 2, wherein the solid material further comprises a lozenge.

4. The mouthguard of claim 2, wherein the gelatinous material further comprises an antiseptic gel.

5. The mouthguard of claim 1, wherein the porous semipermeable membrane comprises a mesh.

6. The mouthguard of claim 1, wherein the reservoir is a plurality of reservoirs.

7. The mouthguard of claim 1, wherein the exterior channel surface defines a groove, optionally wherein the reservoir has a modular attachment to connect with the groove.

8. The mouthguard of claim 1, wherein the channel is shaped to receive the upper teeth or lower teeth of the wearer.

9. The mouthguard of claim 1, wherein the channel is heat moldable to the teeth of the wearer.

10. The mouthguard of claim 1, wherein the porous semipermeable membrane is formed of a material that allows fluid communication between the active component and salivary fluid.

11. The mouthguard of claim 1, wherein the reservoir is formed of a semi-rigid material.

12. The mouthguard of claim 1, wherein the channel is formed of a semi-rigid material or thermoplastic.

13. The mouthguard of claim 1, wherein the channel is shaped in a U-shaped arch.

14. The mouthguard of claim 1, wherein the reservoir extends from a portion of the inner wall.

15. The mouthguard of claim 14, wherein the reservoir is shaped and sized to cover at least a portion of the roof of the mouth of the wearer or at least a portion of the area below the tongue of the wearer.

16. The mouthguard of claim 14, wherein the portion is a first portion, and the reservoir extends from the first portion of the inner wall to a second portion of the inner wall.

17. The mouthguard of claim 1, wherein the reservoir is disposed on a suspension mesh, the suspension mesh connected on the exterior channel surface from a first portion of the inner wall to a second portion of the inner wall.

18. The mouthguard of claim 1, wherein when the holding element is engaged, the active component is held in the interior volume of the reservoir.

19. The mouthguard of claim 1, wherein the holding element comprises a seal.

20. The mouthguard of claim 1, wherein the holding element is disposed to seal the opening when engaged.

21. The mouthguard of claim 1, wherein the holding element comprises a means for providing mechanical pressure to the opposing sides of the opening together.

22. A mouthguard comprising:
a body comprising:
an inner wall,
an outer wall,
a floor spanning the inner wall and outer wall, wherein the inner wall, the outer wall, and the floor define a channel; the channel including an interior channel surface and an exterior channel surface,
the interior channel surface shaped to receive a plurality of teeth; and
a reservoir comprising:
a first layer connected on the exterior channel surface from a first portion of the inner wall to a second portion of the inner wall,
the first layer defining an interior volume for an active component,
the first layer including a porous semipermeable membrane and an opening.

23. The mouthguard of claim 22, wherein the first layer is shaped and sized to cover at least a portion of a roof of the mouth of the wearer or at least a portion of the area below a tongue of the wearer.

24. The mouthguard of claim 22, wherein the first layer defines an interior volume between a roof of the mouth and the layer.

25. The mouthguard of claim 22, the reservoir further comprising:
a second suspension mesh, the second layer connected on the exterior channel surface from the first portion of the inner wall to the second portion of the inner wall, wherein the first layer and the second layer define an opening for receiving an active component, optionally wherein the second layer is disposed to be about parallel to the first layer.

26. A mouthguard comprising:
a body comprising:
an inner wall,
an outer wall,
a floor spanning the inner wall and outer wall, wherein the inner wall, the outer wall, and the floor define a channel; the channel including an interior channel surface and an exterior channel surface,
the interior channel surface shaped to receive a plurality of teeth; and
a reservoir integral with the inner wall, the reservoir comprising a roof portion and a base portion defining an internal volume for an active component, the roof portion comprising an opening for receiving the active component and the base portion comprising one or more openings.
